(19) United States
(12) Patent Application Publication
(10) Pub. No.: US 2018/0000624 A1
(43) Pub. Date: Jan. 4, 2018

(54) DIGIT PROTECTOR DEVICE

(71) Applicant: Valerie Susan MASON, Littleton, MA (US)

(72) Inventor: Valerie Susan MASON, Littleton, MA (US)

(21) Appl. No.: 15/547,463

(22) PCT Filed: Jan. 30, 2016
(86) PCT No.: PCT/US16/15847

§ 371 Date: Jul. 28, 2017

Related U.S. Application Data
(60) Provisional application No. 62/112,500, filed on Feb. 5, 2015.

(51) Int. Cl. A61F 5/01; A61F 5/058; A43B 23/08

(52) U.S. Cl. CPC A61F 5/0195; A61F 5/058; A43B 23/087

(57) ABSTRACT

Disclosed herein is a digit protector device for a foot, toe, hand, and/or finger, which digit protector device, in one embodiment, can be inserted or embedded in the plaster cast for a hand or foot, wrapped into a hand or foot splint bandage, or inserted in an orthopaedic hand or foot brace, boot, or sandal.

This application claims priority to the Provisional Patent Application No. 62/112,500 filed on 5 Feb. 2015, the teachings of which are incorporated herein by reference in their entireties.

### BACKGROUND OF THE INVENTION

Limbs, hands, or feet that are injured or broken are often wrapped in braces, splints or casts, or donned with protective braces or boots until the injuries heal. In order to prevent moisture build up from perspiration, and to aid the healing process, in many cases the fingers or toes of an injured individual are not enclosed, but rather are left exposed and protruding from the cast or splint.

These casts or splints are unwieldy, and reduce limb, finger, and/or toe flexibility and mobility. In addition, the long duration of the healing process can lead to injury to protruding and exposed toes and fingers. Unless the braces, casts or splints are extended to cover at least a portion of the tips of protruding toes or fingers, these digits are susceptible to further injury. However, extending a brace or cast to cover a part of a toe or finger, e.g., would typically be cumbersome and awkward for the patient. Also, it would retard circulation of air needed for healing.

Further, when the cast or splint is removed, following sufficient healing of the injured foot or hand, without a protective device, re-injury can occur.

Currently available toe protector devices are configured to directly contact at least a portion of the foot or hand itself, which can cause friction between the patient's skin and the device and subsequent abrasion of tissue, leading to sores and infection. Some available open-front orthopaedic boots and shoes are designed to relieve heel pressure, but do not afford any protection for the front of the foot, for example, toes. Similarly, a bandaged hand is often left with no protection for protruding fingers.

### SUMMARY OF THE INVENTION

In accordance with the present invention there is provided a device and method that can protect the front portion of a hand or a foot, including digits such as fingers and toes protruding from a cast or brace, and also eliminate the above-described problems by providing an easily insertable or removable protective shield that is not in direct contact with the hand or foot. The inventor of the disclosed subject matter has now developed a digit protector device for use not only with a cast or brace, but that can be used to protect against further injury following removal of the cast or brace. An embodiment of the disclosed device can also be used as a prophylactic against injury in the first place.

Disclosed herein is a digit protector device for a foot, toe, hand, and/or finger, which digit protector device, in one embodiment can be removably inserted into or embedded in an appendage-stabilizing device, such as, for example, a plaster cast for a hand or foot, or wrapped into a hand or foot splint bandage, or inserted in an open-front orthopaedic hand or foot brace, boot, shoe, or sandal.

One embodiment of the invention is an independent digit protector insert for use by a patient to prevent direct contact of at least a portion of a foot or a hand with an object other than a bandage, a lining of an open-front boot or shoe or a cast or splint or lining thereof, the digit protector insert comprising:

a rigid or semi-rigid contoured member having a top surface and a bottom surface;
an elongated, substantially flat proximal end portion of the contoured member configured for embedding under the lining of the open-front boot or shoe or embedding in the cast or splint or the bandage, and for extending at least partially under said foot or hand;
a forward distal end portion having a curvature portion that extends upwards and back toward a top of said foot or hand without touching said foot or hand, the curvature defining a channel configured to receive a portion of said foot or hand protruding from the boot, the shoe, the bandage, the cast, or the splint, the forward distal end portion forming a protective barrier over at least part of the channel,
wherein said curvature does not completely enclose a portion of said foot or hand; and wherein in use by the patient, said digit protector insert configured to be at least partially embedded under the lining of the open-front boot or shoe or embedded in the bandage, the cast, or the splint, prevents direct contact of at least a portion of said foot or hand with an object other than the bandage, the lining of the boot or shoe or the cast or splint or lining thereof, and the forward distal end portion forms a protective barrier over at least part of said foot or hand.

Another embodiment of the invention is an independent digit protector insert for use by a patient to prevent direct contact of at least a portion of a foot or a hand with an object other than a bandage, a lining of an open-front boot or shoe or a cast or splint or lining thereof, the digit protector insert comprising:

a rigid or semi-rigid contoured member having a top surface and a bottom surface;
an elongated, substantially flat proximal end portion of the contoured member configured for embedding under the lining of the open-front boot or shoe or embedding in the cast or splint or the bandage, and for extending at least partially under said foot or hand;
a forward distal end portion having a curvature portion that extends upwards toward a top of said foot or hand without touching said foot or hand protruding from the boot, the shoe, the bandage, the cast, or the splint, the forward distal end portion forming a protective vertical barrier, and
wherein in use by the patient, said digit protector insert configured to be at least partially embedded under the lining of the open-front boot or shoe or embedded in the bandage, the cast, or the splint, prevents direct contact of at least a portion of said foot or hand with an object other than the bandage, the lining of the boot or shoe or the cast or splint or lining thereof.

The disclosed insert provides a number of advantages. It allows air to circulate around the fingers or toes that it is protecting. In contrast to prior art devices, the disclosed insert device, in use by a patient, does not directly contact the hand or foot itself, and thus does not cause friction or abrasion of tissue. In the embodiment for an open-front orthopaedic boot or shoe, the sole of the patient's foot is in contact only with the liner or foot-bed of the shoe or boot. In the embodiment embedded in a cast or a splint, the appendage, e.g., a hand or foot itself, for the portion of the appendage that is embedded in the cast, bandage, or boot sole, does not touch the device, but instead is in contact with only the cast, bandage, or boot liner or inner sole. Because it is embedded beneath the inner sole of the boot or embedded in the cast, the device not only avoids friction with the skin, but also affords greater protection than that of an externally attached device.

In contrast to prior art devices, use of the disclosed device embedded or inserted in a commercially available heel-pressure relieving open-front boot or shoe, allows the boot or shoe to cushion the sole and heel of the foot, reducing pressure on the sole or heel of the foot. An embodiment of the disclosed device will allow the heel pressure-relieving functional design of the commercially available heel relief shoe or boot to relieve heel pressure, while protecting the at least partially exposed toes in the front of the shoe or boot. The disclosed device can protect any digits exposed in the front of the shoe or splint, e.g., in which the device is embedded or inserted.

In contrast to prior art devices, an embodiment of the invention, in use, can support the entire length of the appendage, e.g., the whole limb, foot, hand, or digit. The disclosed protector insert can also include an arch support.

An embodiment of the protector insert is configured independent of an attachment means to an open front boot or shoe. There is no need or requirement for a fastening device to attach the disclosed device to the hand, foot, or other appendage. The prior art appendage-protectors require fastening devices, which suffer from at least the disadvantage of rendering the prior art devices difficult to insert into or embed in an orthopaedic shoe or boot or a cast or splint or a bandage. Modifying one of the prior art designs by removing the fastening device would change the principle of operation of the prior art device.

As will be better understood from the drawings and the claims, the substantially upright wall portion of the distal portion of the disclosed device, inserted or embedded in an open-front orthopaedic shoe or boot or embedded in a cast or splint or a bandage, can also protect a surgical pin or wire protruding from the front of a toe or finger. In an embodiment of the invention having apertures in the distal front vertical wall, the apertures provide an easy access to the surgeon for removal of surgical pins. This feature is especially important in a case wherein the protector device is embedded in a cast and cannot be removed for pulling the surgical pinout without removing the entire cast.

### BRIEF DESCRIPTION OF THE DRAWINGS

Embodiments of the invention are further described hereinafter with reference to the accompanying drawings, in which:

FIG. 1 is a perspective view of a digit protector device according to an embodiment of the invention.

FIG. 2 is a perspective view of a portion of digit protector device according to an embodiment of the invention, the elongated proximal end portion of the device shown partially inserted between the inner and outer soles of an orthopaedic boot.

FIG. 3 is a perspective view of a digit protector device according to an embodiment of the invention, the elongated proximal end portion of the device fully inserted between the inner and outer soles of an orthopaedic boot.

FIG. 4 is a top plan view of a digit protector device according to an embodiment of the invention inserted in an orthopaedic boot and providing protection for the toes and foot.

FIG. 5 is a perspective view of a digit protector device according to an embodiment of the invention embedded in a bandage or placed under a bandaged hand and providing protection for fingers.

FIG. 6A is a side elevational cut-away view of a digit protector device according to an embodiment of the invention embedded in a sole of an open-front shoe or boot.

FIG. 6B is a side elevational cut-away view of a digit protector device according to an embodiment of the invention embedded in a sole of an open-front shoe or boot and depicting a surgical pin protruding from the patient's toe, the pin protected by the digit protector device.

FIG. 6C is a perspective view of a digit protector device according to an embodiment of the invention, the elongated, substantially flat proximal end portion having an hour-glass shape, and a forward distal end portion having a curvature portion that extends upwards.

### DETAILED DESCRIPTION

A description of preferred embodiments of the invention follows. It will be understood that the particular embodiments of the invention are shown by way of illustration and not as limitations of the invention. At the outset, the invention is described in its broadest overall aspects, with a more detailed description following.

The inventor of the present subject matter has discovered a device and method for providing a means for improving the protection provided by currently available medical appendage-stabilizing devices and/or non-medical appendage-stabilizing devices. Non-limiting examples of a medical appendage-stabilizing device for use with an embodiment of the invention include an orthopaedic boot, sandal, hand or foot brace, a plaster hand or foot cast, semi-rigid, removable walking casts, and a hand or foot splint bandage. Non-limiting examples of a non-medical appendage-stabilizing device include a regular shoe, a sandal, a glove, and a sleeve.

The material used to construct the digit protector device can be chosen from any material having the ability to withstand a compressive force of a pre-determined magnitude. In a preferred embodiment, the digit protector device comprises a rigid or semi-rigid material. In one embodiment, the rigid or semi-rigid material is a metal, e.g., steel or aluminum. In another embodiment, the rigid or semi-rigid material material is polycarbonate or a plastic. Fluorinated ethylene propylene resin (FEP) is a highly inert plastic, and resistance to impact and tearing. Almost any FEP or FEP-encapsulated or coated elastomeric material is suitable for use in constructing the disclosed device.

In yet another embodiment, the disclosed device comprises an elastomer or an elastomeric material. An “elastomer” or an “elastomeric material,” as these terms are used and understood by those of skill in the relevant arts is any one of a various polymers having the elastic properties of natural rubber. The disclosed digit protector device may also be constructed of a resilient material. “Resilient,” as the term is used herein, refers to that property of a material that enables it to resume its original shape or position after being stretched, compressed, or bent. Elastomeric and resilient, as the terms are used herein, have similar meanings. The use of any type of rigid or non-rigid material for constructing at least a portion of the disclosed device is also possible. The disclosed device may be constructed of a completely rigid material.

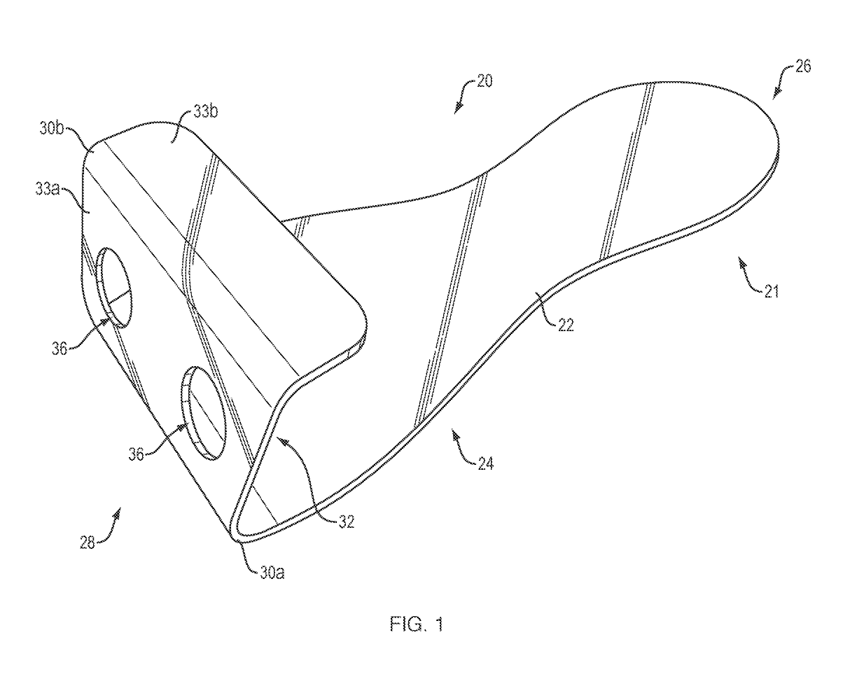

Reference is now made to FIG. 1, which discloses one embodiment of the disclosed digit protector device 20 in the form of a substantially flat or contoured base member 21 configured for placement thereon of an appendage-stabilizing device, base member 21 having a top surface 22 and a bottom surface 24, an elongated proximal end portion 26 configured for attachment to the appendage-stabilizing device, and a distal end portion 28 having a curvature 30a forming a substantially upright wall portion 33a and a curvature 30b forming a substantially horizontal wall portion 33b, the wall portion 33a and the wall portion 33b defining a channel 32 configured to receive at least one digit protruding from the appendage-stabilizing device, the wall portions 33a and 33b forming a protective barrier over at least part of the channel 32, thereby providing protection for the at least one digit when the digit protector device is in use. Shown also in FIG. 1 are optional openings 36 formed in wall 33a, the openings 36 providing ventilation for the front part of the foot and the digits.

As the terms are used herein, “proximal end portion” is the portion of the disclosed digit protector device intended to be place nearer to the center of the body, for example the spinal column; and “distal end portion” is the portion of the disclosed device to be positioned farther from the center of the body.

In the embodiment depicted in FIG. 1, wall 33a and wall 33b are integral with base member 21. In other embodiments (not shown), wall 33a and wall 33b are formed separately from the same or different materials and bonded or otherwise fastened to one another, and wall 33a is bonded or fastened to base member 21.

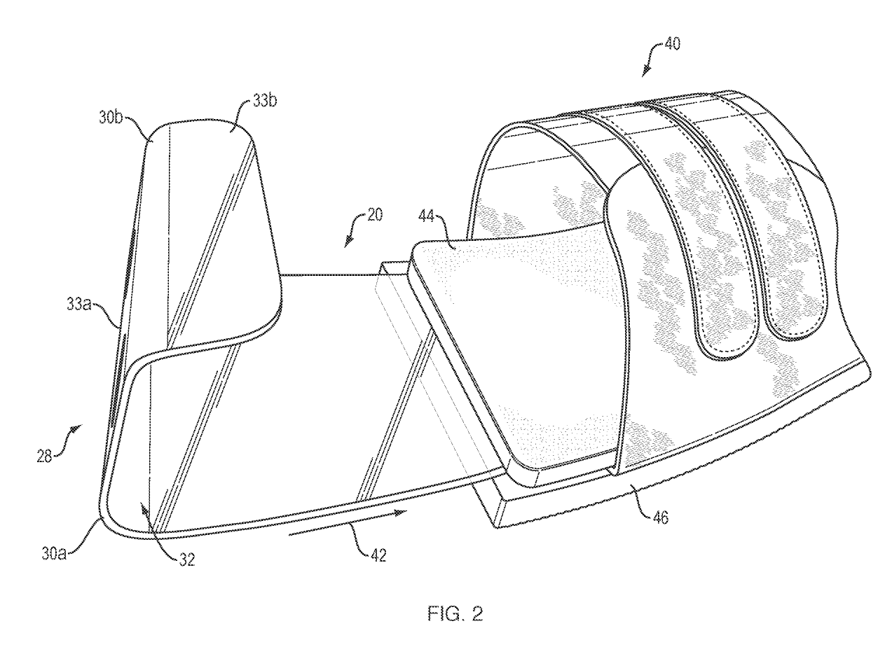

FIG. 2 shows the disclosed digit protector device 20 being inserted in the direction shown by arrow 42 between the upper sole or lining 44 and the lower sole 46 of orthopaedic boot 40.

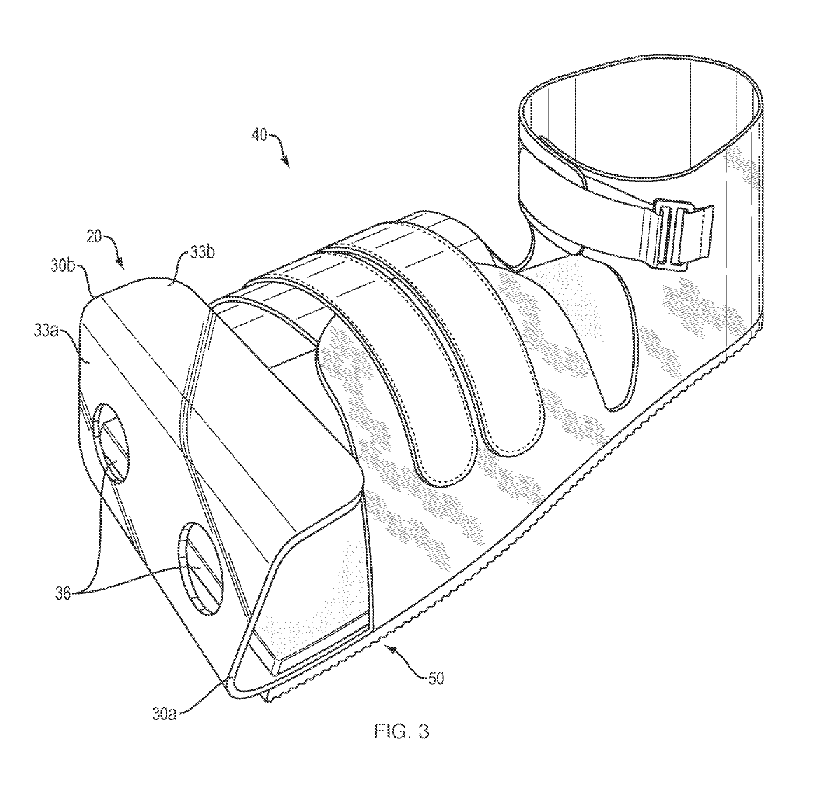

FIG. 3 shows the disclosed digit protector device 20 fully inserted 50 into the orthopaedic boot 40.

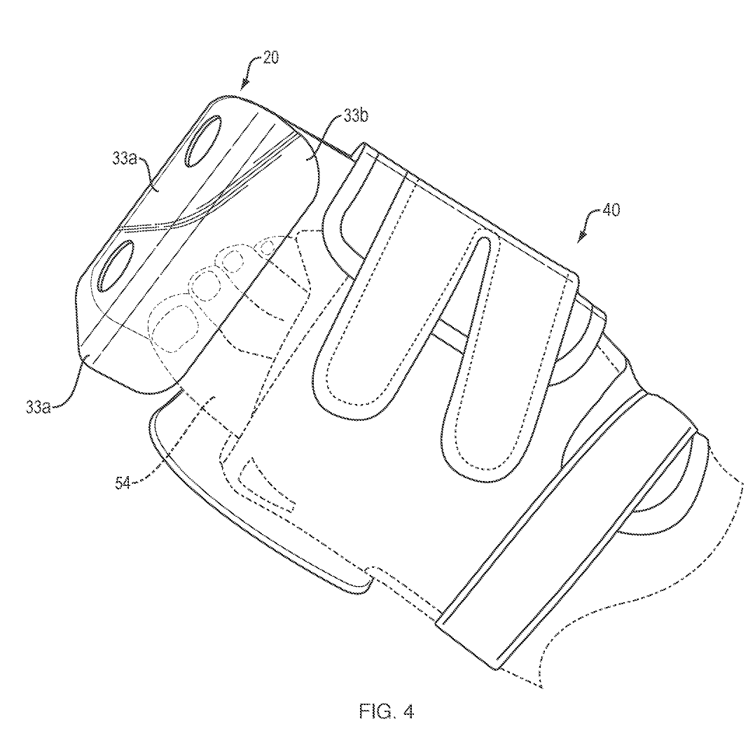

FIG. 4 shows a front portion of the disclosed digit protector device 20 fully inserted in orthopaedic boot 40 and in use on a patient and providing protection for toes 54 and foot.

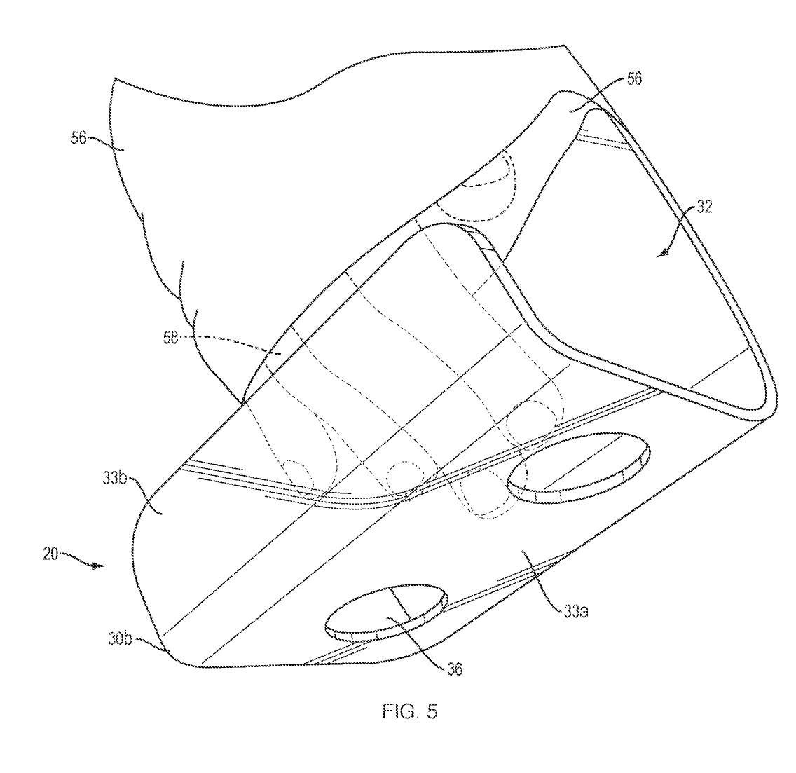

In another embodiment the disclosed device 20 can be included in the preparation of a splint, brace or bandage 56, for example, as shown in FIG. 5. FIG. 5 shows the disclosed digit protector device 20 embedded in a bandage 56 or placed under a bandaged hand and providing protection for fingers 58, while preventing direct contact of a pre-selected portion of the hand with the protector device 20.

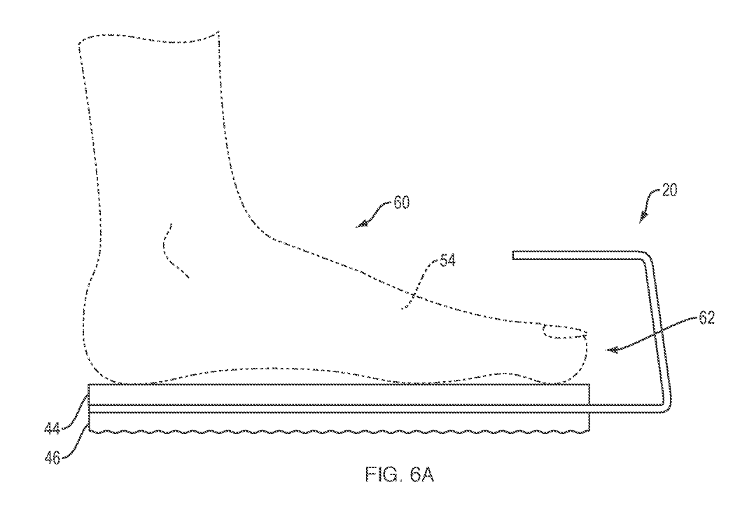

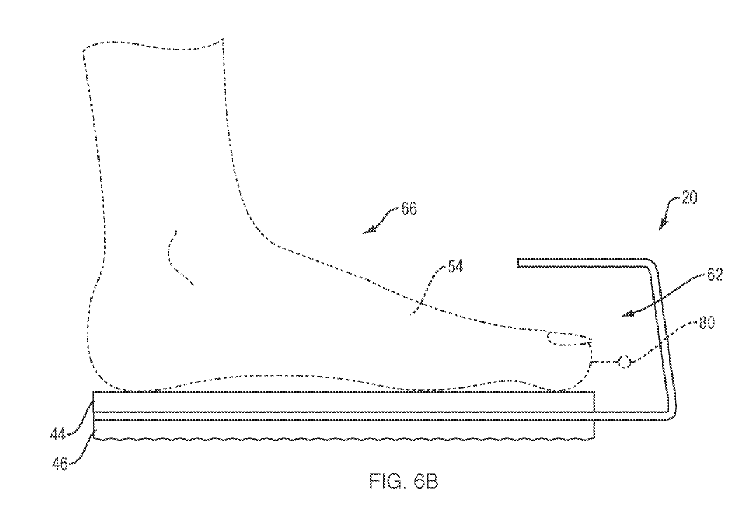

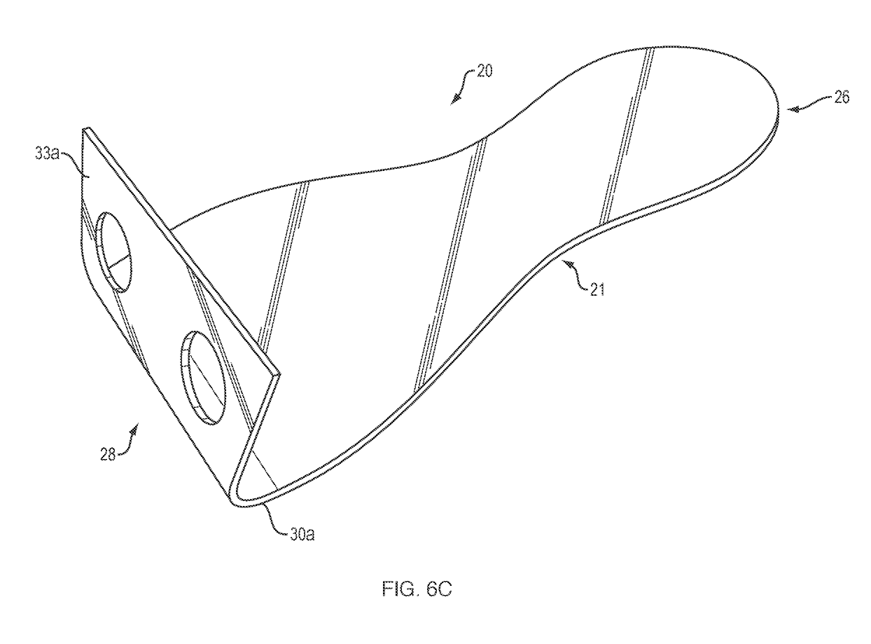

To more clearly depict some aspects of the protection provided by the disclosed device. FIG. 6A is a side elevational cut-away view 60 of a digit protector device 20 according to an embodiment of the invention embedded in a sole of an open-front shoe or boot. FIG. 6A is not drawn to scale. FIG. 6A shows the device 20 “sandwiched” between the lining or inner sole 44 of an orthopaedic shoe or boot and the outer sole 46 of the boot. It is important that the device 20 is anchored or secured under the inner sole or lining 44 such that an impact to the front vertical portion 33a of the insert device 20 does not push the insert device 20 against the toe or finger. In most applications, the elongated proximal end portion 26 of the insert device 20 should be pressed or butted against the inside back of the shoe or the boot. The posterior portion of lining 44 may be an elastomeric or “rubber” type material, such as found in a BAUERFEIND® GLOBOPED™ (Bauerfeind AG, Zeulenroda, Germany) Heel Relief Shoe or boot.

The elastomeric “posterior “lip” of the GLOBOPED orthopaedic boot helps to prevent or to heal heel sores in patients. Such sores can become seriously infected and lead to general sepsis and death. The disclosed insert device 20 works as an insert with this type of boot to allow the elastomeric posterior lip of the orthopaedic boot to function as designed, while the insert device 20 protects the front part of the foot and toes 54 of the patient. In a type of boot having an open heel portion as well as the open front, it is especially important that insert device 20 be secured in place or anchored down. Space 62 provides for circulation of air to promote healing.

FIG. 6B is a partial schematic of a side elevational cut-away view 66 of a digit protector device 20 according to an embodiment of the invention embedded in a sole of an open-front shoe or boot. In order to alleviate pressure on the heel, a particular boot may have a gel cushion or an elastomeric material in the boot positioned to be under the heel of the wearer. Other non-limiting examples of orthopaedic shoes or boots that may be modified for use with the disclosed insert may be available from DARCO® by ALIMED® (Dedham, Mass.).

FIG. 6B also shows a surgical pin 80 inserted in a toe 54 of the patient. In any embodiment of the invention, sufficient space 62 can be provided to accommodate a surgical pin protruding from a toe or a finger. Optional openings 36 (shown in FIGS. 1, 3, 4, 5, and 6C) provided in the substantially upright wall portion 33a of the protector device 20 allow easy access to a surgeon or other medical practitioner for removing the surgical pin without having to remove the protector device 20.

FIG. 6C depicts an embodiment of the insert device 20 having a forward distal end portion having a curvature portion 33a that extends upwards but has no horizontal wall portion 33b as in FIG. 1. FIG. 6C also depicts an hour-glass shaped elongated proximal end for ease of insertion in a shoe or boot. The elongated proximal end can be any shape suitable for insertion in a particular appendage-stabilizing device, and the size can be varied according to the wearer.

In another embodiment, the edges of the elongated proximal end portion 26 of the digit protector device 20 may be serrated or asymmetrically shaped so as to provide better anchoring of the digit protector device in the cast, brace, splint, bandage, or boot.

In yet another embodiment the digit protector device 20 can be fitted with labels or decoration to personalize or to identify the owner or to represent or display a special interest of the owner.

### EQUIVALENTS

Throughout the description and claims of this specification, the words “comprise” and “contain” and variations of them mean “including but not limited to”, and they are not intended to (and do not) exclude other variations, components, or steps. Throughout the description of this specification, the singular encompasses the plural unless the context otherwise requires. In particular, where the indefinite article is used, the specification is to be understood as contemplating plurality as well as singularity, unless the context requires otherwise.

Features, characteristics, or devices described in conjunction with a particular aspect, embodiment or example of the invention are to be understood to be applicable to any other aspect, embodiment or example described herein unless incompatible therewith. All of the features disclosed in this specification (including any abstract and drawings), and/or all of the steps of any method so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying abstract and drawings).

What is claimed is:

1. An independent digit protector insert for use by a wearer to prevent direct contact of at least a portion of a foot or a hand with an object other than a bandage, a lining of an open-front boot or shoe or a cast or splint or lining thereof, the independent digit protector insert comprising:
a rigid or semi-rigid contoured member having a top surface and a bottom surface,
an elongated, substantially flat proximal end portion configured for embedding, anchoring, or securing under the lining of the open-front boot or shoe, or embedding, anchoring, or securing in the cast or splint or the bandage, and for extending at least partially under said foot or hand;
a forward distal end portion having a curvature portion that extends upwards and back toward a top of said foot or hand without touching said foot or hand, the curvature defining a channel configured to receive a portion of said foot or hand protruding from the boot, the shoe, the bandage, the cast, or the splint, the forward distal end portion forming a protective barrier over at least part of the channel,
wherein said curvature does not completely enclose a portion of said foot or hand; and
wherein in use by the wearer, said independent digit protector insert configured to be at least partially embedded, anchored, or secured under the lining of the open-front boot or shoe, or embedded, anchored, or secured in the bandage, the cast, or the splint, such that an impact to the front vertical portion of the independent digit protector insert does not push said digit protector insert against at least a part of said foot or hand;
prevents direct contact of at least a portion of said foot or hand with an object other than the bandage, the lining of the boot or shoe or the cast or splint or lining thereof; and the forward distal end portion forms a protective barrier over at least part of said foot or hand.

2. The independent digit protector insert of claim 1, wherein said independent digit protector insert is configured independent of an attachment means to said open toe boot or shoe.

3. The independent digit protector insert of claim 1, wherein the curvature portion of the forward distal end portion defines at least one opening that allows access to said foot or hand and/or circulation of air to promote healing.

4. The independent digit protector insert of claim 1, wherein the curvature is configured to be sufficiently distal from an end of said foot or hand to accommodate a surgical pin protruding from said end of said foot or hand to allow access to the surgical pin and to allow air flow to circulate in the channel to aid in healing.

5. The independent digit protector insert of claim 4, wherein the curvature portion of the forward distal end portion defines at least one opening that enables a medical practitioner to remove the surgical pin without a need to remove the protector insert from said foot or hand.

6. The independent digit protector insert of claim 1, wherein said independent digit protector insert comprises a plastic material.

7. The independent digit protector insert of claim 1, wherein said independent digit protector insert comprises an elastomeric material.

8. The independent digit protector insert of claim 1, wherein said elongated, substantially flat proximal end portion comprises an hour-glass shape.

9. The independent digit protector insert of claim 1, wherein said elongated, substantially flat proximal end portion comprises an arch support.

10. An independent digit protector insert for use by a wearer to prevent direct contact of at least a portion of a foot or a hand with an object other than a bandage, a lining of an open-front boot or shoe or a cast or splint or lining thereof, the independent digit protector insert comprising:
a rigid or semi-rigid contoured member having a top surface and a bottom surface,
an elongated, substantially flat proximal end portion configured for embedding, anchoring, or securing under the lining of the open-front boot or shoe, or embedding, anchoring, or securing in the cast or splint or the bandage, and for extending at least partially under said foot or hand;
a forward distal end portion having a curvature portion that extends upwards toward a top of said foot or hand without touching said foot or hand protruding from the boot, the shoe, the bandage, the cast, or the splint, the forward distal end portion forming a protective vertical barrier that, when in use by the wearer, is capable of preventing direct contact of at least a portion of said foot or hand with an object other than the bandage, the lining of the boot or shoe or the cast or splint or lining thereof; and
wherein in use by the wearer, said independent digit protector insert configured to be at least partially embedded, anchored, or secured under the lining of the open-front boot or shoe or embedded, anchored, or secured in the bandage, the cast, or the splint, such that an impact to the front vertical portion of the independent digit protector insert does not push said digit protector insert against at least a part of said foot or hand.

11. The independent digit protector insert of claim 1, wherein said elongated, substantially flat proximal end portion is capable of being pressed or butted against the back of the boot or shoe.

12. The independent digit protector insert of claim 10, wherein said elongated, substantially flat proximal end portion is capable of being pressed or butted against the back of the boot or shoe.
